(19) United States
(12) Patent Application Publication
(10) Pub. No.: US 2015/0202465 A1
(43) Pub. Date: Jul. 23, 2015

(54) SOURCE SWITCHING APPARATUS FOR MULTI-SOURCE RADIATION THERAPY EQUIPMENT AND RADIATIONTHERAPY EQUIPMENT USING SAME

(71) Applicant: OUR NEW MEDICAL TECHNOLOGIES, Shenzhen (CN)

(72) Inventor: Xiaofeng Zhao, Xi'an (CN)

(21) Appl. No.: 14/417,771

(22) PCT Filed: Aug. 19, 2014
(86) PCT No.: PCT/CN2014/084705

§ 371 Date: Jan. 27, 2015

(30) Foreign Application Priority Data
Dec. 25, 2013 (CN) CN201320862151.9

(51) Int. Cl. A61N 5/10

(52) U.S. Cl. CPC A61N 5/1077; A61N 2005/1094

(57) ABSTRACT

A source switching apparatus comprises a plurality of collimators, a switch carrier, a source carrier. The switch carrier is assembled with a plurality of shielding elements regularly distributed thereon. The source carrier is assembled with a plurality of radiation sources regularly distributed thereon. The collimator, the switch carrier, and the source carrier are connected to one another via respective driver, to rotate with common angular velocity or in a differential movement, and wherein in the case when the differential movement is holding, a angle difference is formed between the switch carrier and the source carrier.

### CROSS-REFERENCE TO RELATED APPLICATIONS

The present application claims the benefit of PCT application No. PCT/CN2014/084705 filed on Aug. 19, 2014, which is benefit of Chinese patent application No.201320862151.9 filed on Dec. 25, 2013, the disclosure of which is incorporated herein by reference as if reproduced in its entirety.

### TECHNICAL FIELD

The invention relates to a multi-source radiotherapeutic equipment, and more particularly to a source switching apparatus for implementing sealing or unsealing part of radiation sources of a multi-source radiotherapeutic equipment, and a radiotherapeutic equipment.

### BACKGROUND

Currently, multi-source radiotherapy apparatus has become an important tool for treatment of tumors. However, due to the complexity of the human body with various vital organs, it is necessary to avoid some sensitive tissues during treatment. Generally, a way used for avoiding the sensitive tissues is adjusting the position of the body and then changing the angle of radiation incidence, to achieve precise positioning for treatment. For example, patent No. CN1303126712A describes an adjustment device for head. The disadvantage of this approach is a limitation of a range of human activities and the space for treatment, whereby the human body cannot be adjusted freely. As a result, it is difficult to bypass the sensitive tissues during treatment, and finally leading to give up the treatment unexpectedly. In addition, even the body of the patient can be treated, it is not often fixed in a natural position as the body is required not to be moved during treatment. Therefore, the patient can not be in the most comfortable state during treatment. There has been a method to bypass the sensitive tissues during treatment by way of closing part of the radiation sources, such as the approach published in patent No. CN1714884A. However, this method requires a plurality of collimators to accomplish the switching operation, accordingly requires multiple groups of driving mechanisms. As a result, the structure is complex, and the volume is large. Therefore, for an equipment which has a plurality of sources arranged densely, it is difficult to be accomplished while regarding space and structure.

### SUMMARY

In order to solve the above problems, the present invention provides a source switching apparatus for a multi-source radiotherapeutic equipment and a radiotherapeutic equipment using the same. The source switching apparatus includes: a plurality of collimators; a switch carrier, assembled with a plurality of shielding elements regularly distributed thereon; and a source carrier, assembled with a plurality of radiation sources regularly distributed thereon. Wherein the collimator, the switch carrier, and the source carrier are connected to one another via respective driver, to rotate with common angular velocity or in a differential movement, and wherein in the case when the differential movement is holding, an angle difference is formed between the switch carrier and the source carrier.

Preferably, the switch carrier is bowl-shaped.

Preferably, the shielding elements are tungsten rods.

Preferably, the tungsten rods comprise solid tungsten rods and bored tungsten rods.

Preferably, eight groups of shielding elements are regularly distributed on the switch carrier, and correspondingly six groups of radiation sources are regularly distributed on the source carrier.

Preferably, each group of shielding elements comprises 30 shielding elements arranged in six rows.

Preferably, wherein a first group of the eight groups of shielding elements are all bored shielding element, a second group of the eight groups of shielding elements are all solid shielding element, and the other six groups of the eight groups of shielding elements comprise a plurality of bored shielding bodies and a plurality of solid shielding bodies at the same time.

Preferably, each group of the radiation sources comprises five radiation sources.

The present invention further includes a radiotherapeutic equipment, which includes the source switching apparatus as described above.

By use of the source switching apparatus provided in the present embodiment of the invention, a novel method for treating is provided to avoid the sensitive tissues to accomplish radiotherapy treatment for human. This set of source switching apparatus employs the switch carrier with shielding elements only rather than introducing the complex structure of collimators, as such, it is easier to be implemented and the cost of manufacture can be reduced. In addition, with a combination of the source switching apparatus with a conventional one, a wider range of the patient's body can be treated, and the treatment way would be more flexible. Besides, the patient will feel more comfortable during treatment.

### BRIEF DESCRIPTION OF THE DRAWINGS

FIG. 1 is a schematic cross-sectional structure of a source switching apparatus for a multi-source radiotherapeutic equipment, according to an embodiment of the invention.

FIG. 2 is a schematic view of an arrangement of radiation sources on the source carrier which is shown in FIG. 1.

FIG. 3 is a schematic view of an arrangement of tungsten rods on the switch carrier which is shown in FIG. 1.

FIGS. 4 and 5 are schematic diagrams of the application for intracranial lesions treatment, according to an embodiment of the invention.

### DETAILED DESCRIPTION

The technical solutions of the present invention are further described in detail with reference to attached drawings and specific embodiments. Obviously, the embodiments described herein are merely exemplary ones, but are not all the embodiments. Based on the embodiments of the present invention, all other embodiments derived by persons of ordinary skill in the art without any creative efforts shall fall within the protection scope of the present invention.

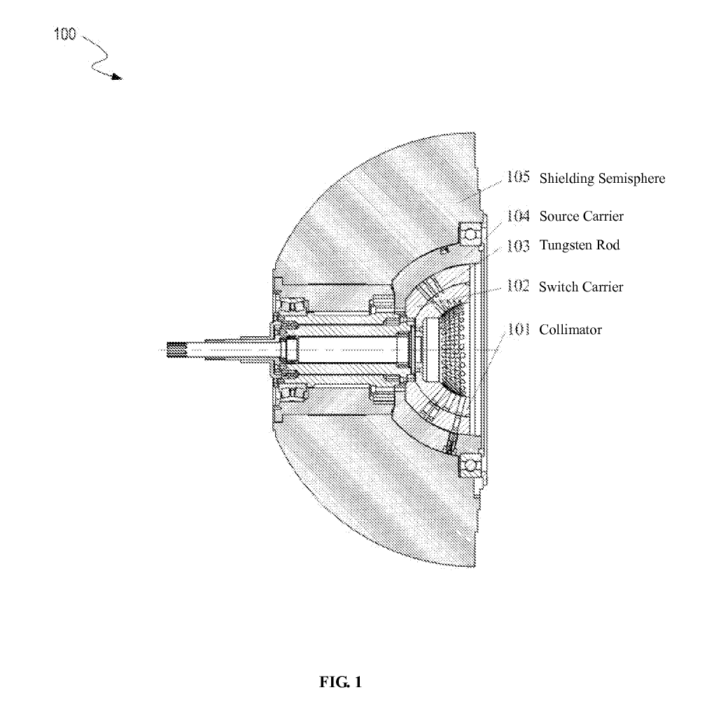

FIG. 1 is a schematic cross-sectional structure of a source switching apparatus for a multi-source radiotherapeutic equipment, according to an embodiment of the invention. The source switching apparatus 100 includes a plurality of collimators 101, a switch carrier 102, a plurality of tungsten rods 103, a source carrier 104, and a shielding portion 105. The appearance of the switch carrier 102 is but not limited to a bowl-shape. The collimator body 101, switch carrier 102, and the source carrier 104 are connected to respective driving mechanism (not shown) through three independent drivers. Thus, a synchronous movement or a differential movement of the three components, namely the collimators 101, the switch carrier 102, and the source carrier 104, can be realized by controlling the driving mechanism. In the case of normal operation, the collimator 101, the switch carrier 102, and the source carrier 104 maintain synchronous movement (i.e., rotate in common angular velocity), and therefore a set of radiation sources disposed on the source carrier 104 are unsealed through one set of holes (for example, a seventh group of tungsten rods all equipped with bored tungsten rods) which are disposed on the switch carrier 102. When it is needed to accomplish closing/sealing part of the radiation sources, the collimators 101, the switch carrier 102, and the source carrier 104 rotate relative to each other (i.e., in differential movement) while increasing or decreasing the velocity thereof At this time, an angle difference are formed between the switch carrier 102 and the source carrier 104, with the set of radiation sources on the source carrier 104 aligning with another set of holes (e.g., a first group of tungsten rods partially equipped with solid tungsten rods) on the switch carrier 102, so that sealing or unsealing partial radiation sources is realized. Advantageously, through the differential movement between the collimators 101, the switch carrier 102, and the source carrier 104, a movement of a relative positions (i.e., changing the angle difference) between the switch carrier 102 and the source carrier 104 can be realized, enabling a function of sealing and unsealing the radiation sources for the multi-source radiotherapeutic equipment. It is understood that although the present embodiment does not specify the size of the angle difference, in operation, those skilled in the art may take advantage of the differential movement between the switch carrier and the source carrier, to achieve any suitable angle differences for partially sealing and unsealing the radiation sources.

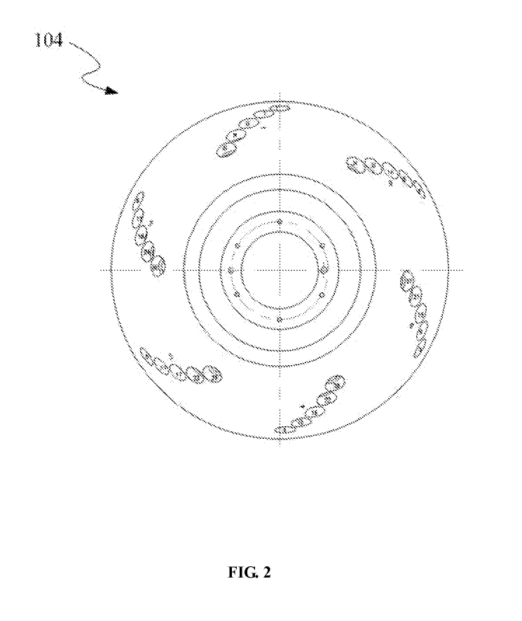

FIG. 2 is a schematic view of the arrangement of the radiation sources disposed on the source carrier 104 shown in FIG. 1. As shown in FIG. 2, a plurality groups of radiation sources (e.g., six groups of radiation sources) are regularly distributed on the source carrier 104, and each group of the radiation sources includes a plurality of radiation sources (e.g., five radiation sources). Note that although the radiation sources are arranged in a specific arrangement with particular number as shown in FIG. 2, it is illustrative only but not limitation. On the contrary, those skilled in the art may take other possible ways to arrange the groups of radiation sources, and the total number of the radiation sources may be more or less than 30.

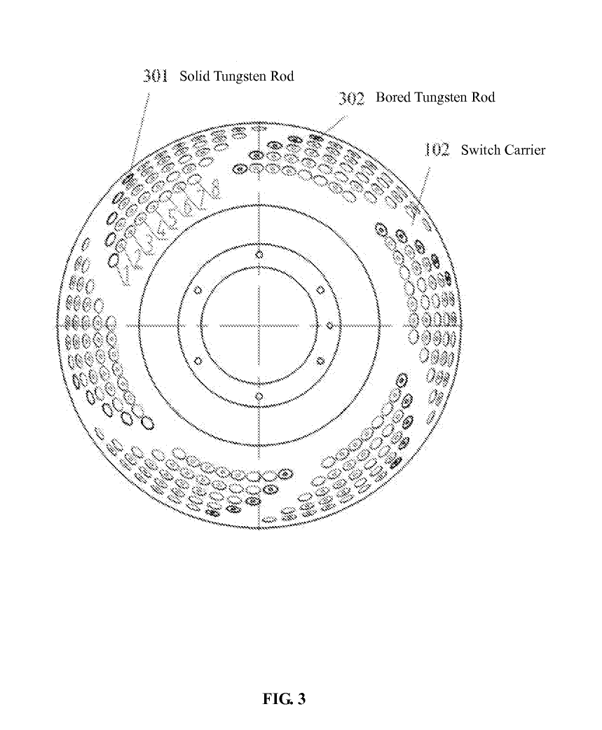

FIG. 3 is a schematic view of the arrangement of shielding elements (e.g. the tungsten rods 103) disposed on the switch carrier 102 shown in FIG. 1. The shielding elements can be classified into solid type shielding elements and bored type shielding elements, such as solid tungsten rod 301 and bored tungsten rod 302. The solid tungsten rod 301 is configured to shield radiation and sealing the radiation source. The bored tungsten rod 302 is configured to pass the radiation and unsealing the radiation source. As shown in FIG. 3, a plurality groups of tungsten rods 103 (e.g., eight groups of tungsten rods) are regularly distributed on the switch carrier 102, and each group of tungsten rods includes a plurality of tungsten rods (e.g., 30 tungsten rods, which is divided into six rows). Note that although the shielding element is described with tungsten rod as shown in FIG. 3, it is illustrative only but not a limitation. On the contrary, those skilled in the art may take other possible shielding material and arrangement for the shielding elements.

In FIG. 3, the specific distribution of the tungsten rods is shown as following: Regarding the first to sixth groups of tungsten rods, two rows of each group of tungsten rods are equipped with solid tungsten rods 301, and the other four rows of each group of tungsten rods are equipped with bored tungsten rods 302. With relative rotation between the switch carrier 102 and the source carrier 104, the six groups of tungsten rods on the switch carrier 102 may correspond to six groups of radiation sources on the source carrier 104. Two groups of the radiation sources cannot transmit radiation out as just aligning against the solid tungsten rods 301, therefore accomplishing the closure/sealing of the two groups of radiation sources. The other four groups of radiation sources are aligned with the bored tungsten rods 302, and accordingly can emit radiation to realize the unsealing operation of these radiation sources. In this way, the function of sealing and unsealing part of the radiation sources for the multi-source radiotherapeutic equipment is accomplished.

Regarding the seventh group of the tungsten rods, all tungsten rods 302 are equipped with bores. Correspondingly, the six groups of radiation sources distributed on the source carrier 104 are in a completely unsealed position.

Regarding the eighth group of the tungsten rods, all tungsten rods are equipped with solid tungsten rods 301. Correspondingly, the six groups of radiation sources arranged on the source carrier 104 are in a completely sealed position.

Advantageously, the six groups of tungsten rods disposed on the switch carrier 102 are arranged according to a specific rule, therefore, sealing the first group to the sixth group of the radiation sources and continuously sealing two adjacent radiation sources can be realized through mutual rotation between the switch carrier 102 and the source carrier 104. Thus, a range of 0°-120° without radiation during rotation can be achieved, thereby the sensitive tissues can be protected.

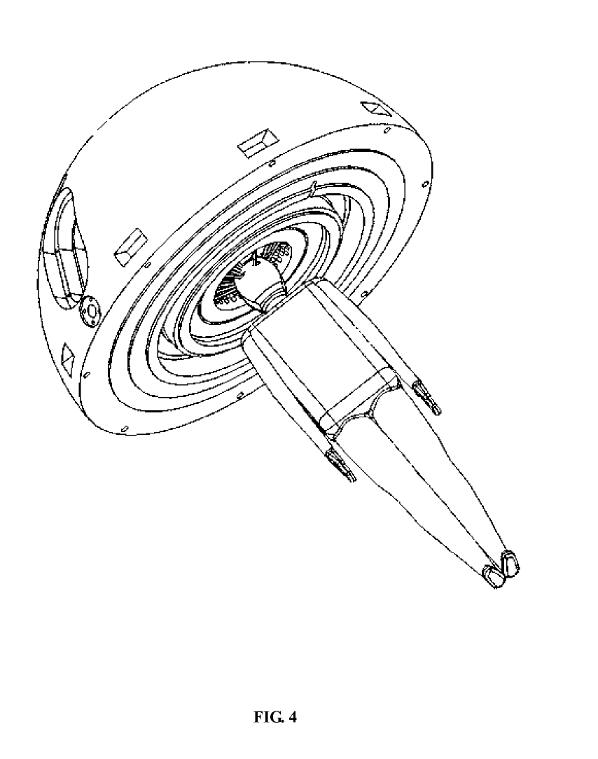

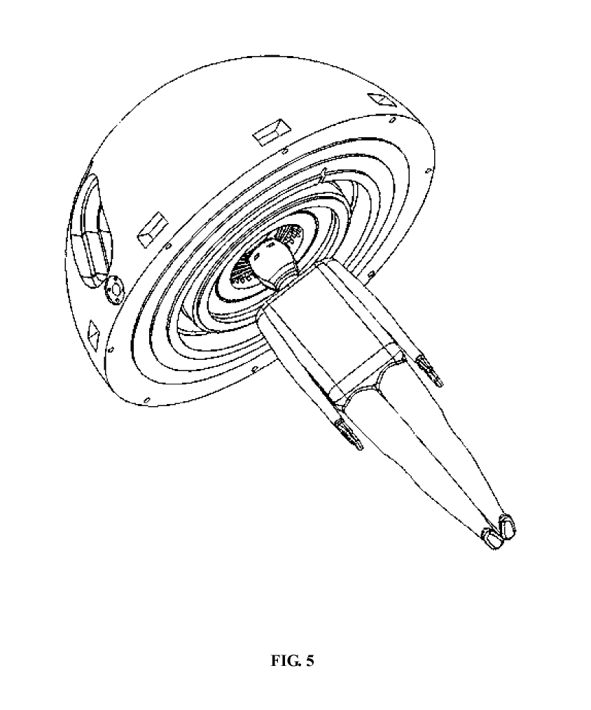

FIGS. 4 and 5 are schematic diagrams of the application of intracranial lesions treatment according to an embodiment of the invention. Preferably, FIGS. 4-5 can be understood in combination of FIGS. 1-3 described above. As shown in FIG. 4, when the radiation sources are all unsealed, there will be a part of radiation rays passes through the eyes of the patient. However, this is not permitted in treatment. At this time, the way of sealing partial radiation sources can be employed. As shown in FIG. 5, through the mutual rotation between the switch carrier 102 and the source carrier 104, it is available to only block the radiation from two groups of radiation sources which will pass through the eyes. As such, it is easy to avoid hurting the eye tissues and accomplish the treatment.

Advantageously, by use of the source switching apparatus provided in the present embodiment of the invention, a novel treating method is provided to avoid the sensitive tissues to accomplish radiotherapy treatment for human. This set of source switching apparatus employs the switch carrier with shielding elements only, rather than introducing a modified and complex structure of collimators. As such, the structure is easier to be implemented and the cost of manufacture can be reduced. In addition, with a combination of the source switching apparatus with a conventional one, a wider range of the patient's body can be treated, and the treatment way would be more flexible. Besides, the patient will feel more comfortable during treatment.

The above descriptions are merely a preferred embodiment of the present invention, but are not intended to limit the present invention. Any modification, equivalent replacement, or improvement made without departing from the spirit and principle of the present invention should fall within the protection scope of the present invention.

1. A source switching apparatus for a multi-source radiotherapeutic equipment, comprising:
a plurality of collimators;
a switch carrier, assembled with a plurality of shielding elements regularly distributed thereon; and
a source carrier, assembled with a plurality of radiation sources regularly distributed thereon,
wherein the collimator, the switch carrier, and the source carrier are connected to each other via respective driver, to rotate to each other with common angular velocity or in a differential movement, and wherein in the case when the differential movement is holding, an angle difference is formed between the switch carrier and the source carrier.

2. The source switching apparatus according to claim 1, wherein the switch carrier is bowl-shaped.

3. The source switching apparatus according to claim 1, wherein the shielding elements are tungsten rods.

4. The source switching apparatus according to claim 3, wherein the tungsten rods comprise solid tungsten rods and bored tungsten rods.

5. The source switching apparatus according to claim 1, wherein eight groups of shielding elements are regularly distributed on the switch carrier, and correspondingly six groups of radiation sources are regularly distributed on the source carrier.

6. The source switching apparatus according to claim 5, wherein each group of shielding elements comprises 30 shielding elements arranged in six rows.

7. The source switching apparatus according to claim 5, wherein a first group of the eight groups of shielding elements are all bored shielding element, a second group of the eight groups of shielding elements are all solid shielding element, and the other six groups of the eight groups of shielding elements comprise a plurality of bored shielding bodies and a plurality of solid shielding bodies.

8. The source switching apparatus according to claim 5, wherein each group of the radiation sources comprises five radiation sources.

9. A radiotherapeutic equipment, comprising a source switching apparatus, wherein the source switching apparatus comprises:
a plurality of collimators;
a switch carrier, assembled with a plurality of shielding elements regularly distributed thereon; and
a source carrier, assembled with a plurality of radiation sources regularly distributed thereon,
wherein the collimator, the switch carrier, and the source carrier are connected to each other via respective driver, to rotate to each other with common angular velocity or in a differential movement, and wherein in the case when the differential movement is holding, an angle difference is formed between the switch carrier and the source carrier.

10. The radiotherapeutic equipment according to claim 9, wherein the switch carrier is bowl-shaped.

11. The radiotherapeutic equipment according to claim 9, wherein the shielding elements are tungsten rods.

12. The radiotherapeutic equipment according to claim 11, wherein the tungsten rods comprise solid tungsten rods and bored tungsten rods.

13. The radiotherapeutic equipment according to claim 9, wherein eight groups of shielding elements are regularly distributed on the switch carrier, and correspondingly six groups of radiation sources are regularly distributed on the source carrier.

14. The radiotherapeutic equipment according to claim 13, wherein each group of the shielding elements comprises 30 shielding elements arranged in six TOWS.

15. The radiotherapeutic equipment according to claim 13, wherein a first group of the eight groups of shielding elements are all bored shielding element, a second group of the eight groups of shielding elements are all solid shielding element, and the other six groups of the eight groups of shielding elements comprise a plurality of bored shielding bodies and a plurality of solid shielding bodies at the same time.

16. The radiotherapeutic equipment according to claim 13, wherein each group of the radiation sources comprises five radiation sources.
